(19) United States
(12) Patent Application Publication
(10) Pub. No.: US 2026/0306364 A1
(43) Pub. Date: Oct. 8, 2026

(54) DEVICE FOR AUTOMATICALLY DISPENSING MEDICATIONS AT PATIENT BEDSIDE IN HOSPITAL CONTEXTS

(71) Applicant: BEPI DISPENSER S.r.I., Trezzano sul Naviglio (MI) (IT)

(72) Inventors: Roberto Schibuola, Trezzano sul Naviglio (MI) (IT); Francesco Tasso, Genova (IT); Cristina Beviacqua, Finale Ligure (SV) (IT)

(73) Assignee: BEPI DISPENSER S.r.I., Trezzano sul Naviglio (MI) (IT)

(21) Appl. No.: 19/485,598

(22) PCT Filed: Mar. 8, 2024
(86) PCT No.: PCT/IB2024/052240

§ 371 Date: Nov. 18, 2025

(30) Foreign Application Priority Data
May 19, 2023 (IT) 102023000010233

(51) Int. Cl. A61J 7/00; A61J 1/03; A61J 7/04

(52) U.S. Cl. CPC A61J 7/0084; A61J 1/035; A61J 7/0427; A61J 2200/74

(57) ABSTRACT

A medication automatic dispensing system in hospital context which allows the preparation of single medication and multi-medication doses for personalized therapies administering, which allows the traceability of the medications contained in the device, together with the therapy administering, and a simple and fast management of the modification of the therapy administered, and allow the traceability of the operations carried out by each health care professional, of the medications loaded and still present in the device and to allow to retrieve all the medications loaded in the device when the patient is discharged.

### BACKGROUND OF THE INVENTION

### 1. Field of the Invention

Aim of the present invention is a medication automatic dispensing system in hospital context which allows the preparation of single medication and multi-medication doses for personalized therapies administering.

### 2. Brief Description of the Prior Art

At the state of the art, many devices are known configured for dispensing medications to patients, both at home and at the hospital.

A first example is a device marketed by Philips (Medication adherence solution, Philips Medication Dispenser), in which a dispenser is configured to receive a preloaded recharge cartridge full of single-dose medication packages, and to remind patients suffering from chronic diseases to take medications at predetermined times. The preloaded medication refills are delivered directly at home of patients from a pharmacy by post. A second device, known on the market with the name “Hero” by Hero Health, allows to charge a plurality of loose, and of a single medication, pills inside a device which then allows the automatized dispensing thereof.

Another example is the device known on the market as PIVOTELL ADVANCE which contains 28 compartments to contain medications, and at predetermined times the user, by turning the device upside down, can extract only the medications which has to be administered at that time. The mechanical configuration is such that the number of loadable medications is reduced, and it is not provided the possibility to share the usage with more than a patient. A similar device is the one known as MED-O-WHEEL or the devices known on the market as Med-Elert's or TUNSTALL MEDICATION DISPENSER.

A device known as MED-MINDER allows the preloaded pills to be dispensed through trays inside the device.

These, and other similar devices known at the state of the art are all optimized to be used at home, and do not allow the automatized dispensing of multi-medication therapies to a plurality of patients in a hospital context.

Also, in the Patent field various embodiments of devices for medication automatic dispensing are described.

US20190130078 and US20210030626 describe devices provided with a single reservoir for medications, so with the impossibility to distinguish between different type of pills.

U.S. Pat. No. 10,292,906 describes a device configured to contain each multi-medication dose inside a single reservoir compartment. This makes impossible both to modify the therapy administered (since it is the preloaded therapy) and trace possible errors of the planned therapy.

Moreover, it does not allow to trace single pills or tablets, for their next usage. Similar limitations are found in the device U.S. Pat. No. 11,166,879.

Similar disadvantages are found in U.S. Pat. No. 8,068,934, which is also mechanically complex. The device is provided with a plurality of loading holes (one for each medication) complicating the loading step for the nurse, thus adding a possible source of error. Moreover, pills are introduced without blisters, thus leading to possible problems of medication storage.

EP2355769 describes a device in which pills are organized in preloaded columns, with the possibility to dispense only the pill in the lowest position in the column and so without the possibility to select the single pill. US20210361531 describes a device for the extraction of pills from their blisters that requires many manual actions by the user (many buttons are provided to cut, move blisters, etc).

U.S. Pat. No. 4,811,764A describes a device that allows access to the storage where the pills are contained only to authorized personnel. To access the storage, it is necessary to have security codes to be entered via a keypad or a magnetic card.

### SUMMARY OF THE INVENTION

None of the medication dispensing devices known at the state of the art allows to obtain at the same time all the following advantages: easiness of charging by the nurse; management of a plurality of medications of different type; management of the therapy of a plurality of patients; traceability of the operations carried out by the nurse and of the correct taking of the therapy by the patients; individual traceability of each single medication, also for a next re-usage; possibility to use medications both loose and inside the blisters; possibility to modify the planned therapy without the need to empty the device.

Ultimately, none of the devices known at the state of the art allows an efficient usage in hospital context, where all these problems become relevant, but they are all substantially limited to an usage exclusively at home. Aim of the invention Therefore, aim of the present invention is a medication automatic dispensing system in hospital context which allows the preparation of single medication and multi-medication doses for personalized therapies administering, which allows the traceability of the medications contained in the device, together with the therapy administering, and a simple and fast management of the modification of the therapy administered.

Aim of the present invention is also to provide a medication automatic dispensing system in hospital context which allows, thanks to a univocal identification of the same, to retrieve all the medications loaded in the device when the patient is discharged.

Yet, another aim of the present invention is to provide a device which allows dispensing of medications both “loose” and contained in their own blisters, possibly pre-cut.

Yet, according to another aim the invention provides a medication automatic dispensing system in hospital context which allows the traceability of the operations carried out by each health care professional, of the medications loaded and still present in the device, of the state of the device and of the dispensing carried out, and of the respect of the intended therapy by each patient.

### Brief Description of the Invention

The present invention reaches the prefixed aims since it is a device for medication automatic storage and dispensing, comprising: a medication storage (10) comprising a plurality of discs (101), each disc (101) comprising a plurality of compartments (1012) open on the top and provided with openable bottom, and a bottomless compartment (1013), said discs (101) being coaxially superimposed on each other and configured to be able to rotate to each other, so that when said bottomless compartments (1013) are superimposed on each other, a medication charge/discharge channel is obtained, passing from the top to the bottom of said storage (10); a case (1) provided with a frontal opening (13) protected by a door (11) through which said medication storage (10) can be introduced inside said case (1), and an upper opening (12), through which the medications can be introduced inside the storage (10); a removable collection container (2) of medications to be dispensed to the patient positioned inside said case, under said medication storage (10) and at said upper opening (12), said door (11) being configured so as to allow, even when it is closed, to extract said container (2) through a medication access area (111); automatic protection means of said medication access area (111) and of said upper opening (12); an actuation system configured to allow the individual and independent rotation of each disc; control means configured to control the opening of said automatic protection means of said medication access area (111) and of said upper opening (12), said actuation system for the discs rotation and the opening of said compartments (1012) provided with openable bottom. Description of the invention

The integrated system according to the invention comprises: at least a dispensing device (automatic dispenser), and preferably a plurality of dispensers, at least a wearable device (preferably a vibrating bracelet), univocally associated to a respective patient,
at least a main control and management electronic device (tablet or hand-held computer or smartphone) for the management of the system.

Preferably, but not limitingly, the system further comprises at least a wearable device (preferably a vibrating bracelet or smartphone) associated to a health care professional.

The system further comprises computer programs, loaded on respective computing electronic means, accessible by means of remote communication, and configured to manage the devices provided in the system.

From a logic point of view, the computer programs are preferably cloud-based (i.e. installed on a remote processing means) and connected by means of a network connection to all the other nodes of the system, according to a “star” architecture. Hence, the data processing will occur at centralized level, with exchange of unilateral (from platform to bracelets) and bilateral (towards dispenser, tablet and applications of third parties) instructions and controls.

In a preferred embodiment the computer programs are configured to allow the following information exchange between the various devices constituting the system.
1. Information exchanged between computing electronic means and dispenser (and vice-versa): therapy: sequence of medications and administering times,
therapy dispensed correctly,
therapy withdrawn correctly from dispenser,
therapy dispensed incorrectly,
therapy withdrawn incorrectly,
discharged battery of dispenser,
other dispenser signals of self-diagnosis to be defined in detail.
2. Information exchanged between computing electronic means and main control and management electronic device therapy planning: sequence of medications and administering times of each patient,
barcode read by the tablet camera or specific barcode reader connected to tablet, for the single medication in the dispenser charging step,
medication corresponding to a determined barcode,
confirmation of successful dispensing,
confirmation of correctly withdrawn therapy,
alarms: therapy dispensed incorrectly to a determined patient,
therapy withdrawn incorrectly by a patient,
discharged battery of a dispenser,
other dispenser signals of self-diagnosis to be defined in detail.
3. Information exchanged between dispenser and each wearable device univocally associated to a respective patient: therapy ready to be withdrawn.
4. Information exchanged between computing electronic means and each wearable device associated to a health care professional
signal of alarm on a dispenser
5. Information exchanged between computing electronic means and applications of third parties:
barcode acquired in a dispenser charging step, medication on the market corresponding to a determined barcode.

Furthermore, it is not to be excluded a priori that some specific data can be provided according to different channels (for example, the national database access application of medications can be directly installed on the tablet of the hospital ward).

Moreover, it is important to notice that, in case the scanned barcode is not present in the national database of medications, the logged nurse will be able with his own credentials to force the introduction of the medication in the dispenser, thus avoiding the procedure interruption in case the national database is not completely updated.

The computer programs managing the system according to the invention are also configured to carry out the medication charging, storing, dispensing and discharging steps according to the procedures shown in the following.

(1000) Medication charging and storing step (1010) Associating patients: at least a wearable device associated to a respective patient is associated to a device.

(1020) Creating the prescription: upon authentication by means of username and password, a health care professional inputs in the system, through the main control and management electronic device, the data relating to the therapy to be followed by each patient associated to the device in step (1010), as a function of the medical prescription, indicating for each medication each patient has to take:
medication name,
dose to be taken,
administering frequency.

(1020) Computing the total needed medications quantities to be charged in the device as a function of the prescriptions input in step (1020), (1030) charging each medication in the device, according to the specific procedure described in the following.

(2000) Dispensing Step.

At the administering time of a medication to a patient, when the digital platform detects that the therapy dispensing time has arrived for a certain patient, it activates remotely the dispenser of that patient.

The dispenser, through its inner control system, activates the dispensing operations of the first medication of the multi-medication dose. The sensoristics inside the dispenser detects
a. if the medication has been correctly dispensed within a certain time interval. If the medication has not been correctly dispensed, an error message is displayed on the user interface in the tablet and at the same time the ‘nurse’ s bracelet vibrates. The nurse, after checking on the tablet the type of the problem, will have to deal with the therapy administering.
b. If the medication has been correctly dispensed and it is the last medication of the multi-medication dose to be dispensed, the dispenser outputs a light signal of confirmation, and at the same time, the patient ′ s bracelet vibrates. The patient will have to withdraw the whole therapy and take it.
c. If the medication has been correctly administered and it is not the last medication to be dispensed, the medication dispensing continues up to dispensing the last medication. When the whole multi-medication dose has been dispensed, the sensoristics inside the device (see below) checks if the same has been really withdrawn within a certain time interval, and if the container has been really emptied before being repositioned.
d. If the therapy has not been correctly withdrawn, an error message is displayed on the user interface in the tablet and at the same time the nurse's bracelet vibrates. The nurse, after checking on the tablet the type of the problem, will have to deal with the therapy administering. e. If the therapy has been correctly withdrawn, a confirmation message will be sent to said at least main device.

Without this limiting the aims of the invention, the message of successful administering can be sent to a software application managing the medical record, so to record the successful medication administering. In case of lack of connection between the central software and the single devices (for example, for temporary network problems), the system is configured:
to recognize the status of disconnection, to notify the status of disconnection and the duration of disconnection to the staff. On the basis of this information, the staff of the hospital ward can actuate the manual check procedure for the medication administering.

(3000) Discharging Step.

The device is further configured to discharge completely the dispenser, for example after a patient is discharged, allowing anyway to retrieve the medications contained therein, for a next usage thereof so as to avoid waste for the hospital.

To such aim, the management software will provide all the information relating to the single discharged medication (description, quantity, and optionally lot and/or expiry date) and it will be able to be connected to a printer for adhesive labels, which will be used according to the procedure described in the following.
1. The nurse sets on the tablet the discharge function start for a determined dispenser.
2. The managing software sends to the dispenser a discharge instruction of the first medication (medication A), based on the current filling status (which is known since the charging and dispensing steps are traced by the managing software).
3. The managing software sends to the printer the instruction to print a label containing the information about medication A: medication name, quantity and optionally lot and/or expiry date. In case there is no printer (since it is an optional component), the nurse himself will have to compile the label manually, copying the data displayed on the tablet, in case it is needed the medication retrieval.
4. All the positions containing the medication A are discharged one by one by means of the same mechanisms controlling the dispensing. A pill of medication A is discharged at a time until the dispenser is empty.
5. The nurse puts each pill of medication A back in a container (a bag or plastic box), and puts the adhesive label on the container to guarantee the future tracing of the content.
6. Steps 2 to 5 are repeated for all the types of medications (medication B, medication C, etc.) contained in the dispenser.
7. At the end of the procedure, the containers marked by the adhesive labels are stored, in order that they can be re-used, by considering the lots and expiry dates.

### (4000) Therapy at Need

According to a preferred embodiment, the device is configured to allow the patient to ask for the dispensing of a determined medication in a given time, through recognition by bracelet and by pressing a button on the device or other command actuated by the patient on purpose.

According to the different medications, norms and therapeutic prescriptions, the hospital ward staff can set maximum dispensing limits of pills at need within determined time intervals (for example maximum 2 dispensing at need of medication “X” in the 24 h). Each medication dispensing at need will be recorded on the centralized digital platform and notified on the tablet of the hospital ward. Mechanical description of the dispensing device It is now described the preferred embodiment of the device for medication storage and distribution according to the invention, with reference to FIGS. 5 to 17.

### BRIEF DESCRIPTION OF THE DRAWINGS

FIG. 1 shows a preferred embodiment of a scheme of logic connections between the various components of the system.

FIGS. 2, 3, and 4 show functioning schemes of the system in the charging, dispensing and discharging steps.

FIG. 5 shows the disc basic unit of the medication storage;

FIG. 6 shows the medical storage discs stacked on each other;

FIG. 7a and b show two views of the opening system of the trapdoor of the nth disc sector;

FIG. 8 shows a disc actuation system;

FIG. 9 shows the disc extraction system from the case;

FIG. 10 shows a preferred embodiment of the wearable device;

FIG. 11 shows an embodiment of the coupling system to the bed of the patient of the device according to the invention;

FIG. 12 shows a schematic view of the pick and place system and of the displaying system;

FIG. 13 shows a schematic view of the inner mechanical portion of the deblistering module;

FIG. 14 shows a schematic view of the conveying system of deblistered medications inside the introduction channel of the device;

FIG. 15 shows the deblistering device assembled on the dispensing device;

FIG. 16 shows the device in the medication dispensing step, with the container accessible;

FIG. 17 shows a schematic view of the arrangement of the main components inside the case, with the case not show for clarity.

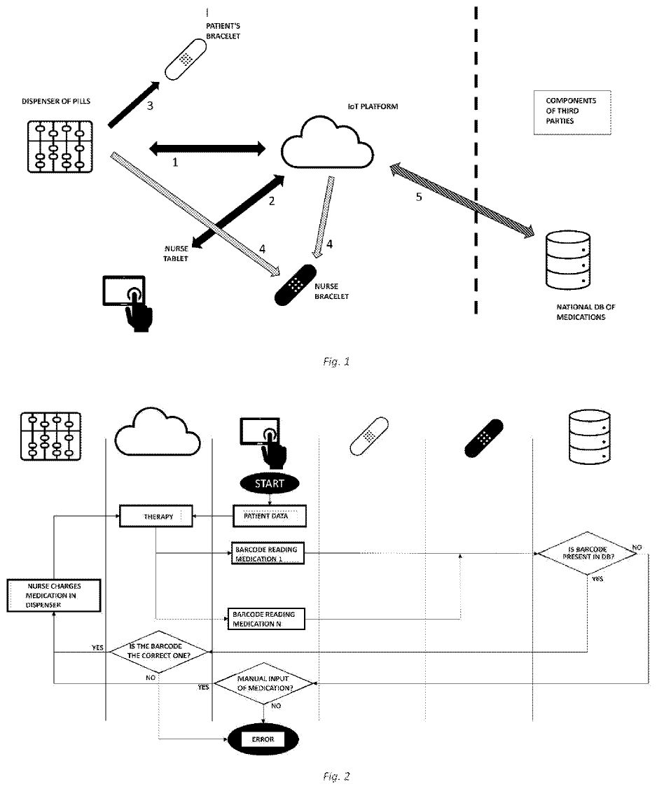

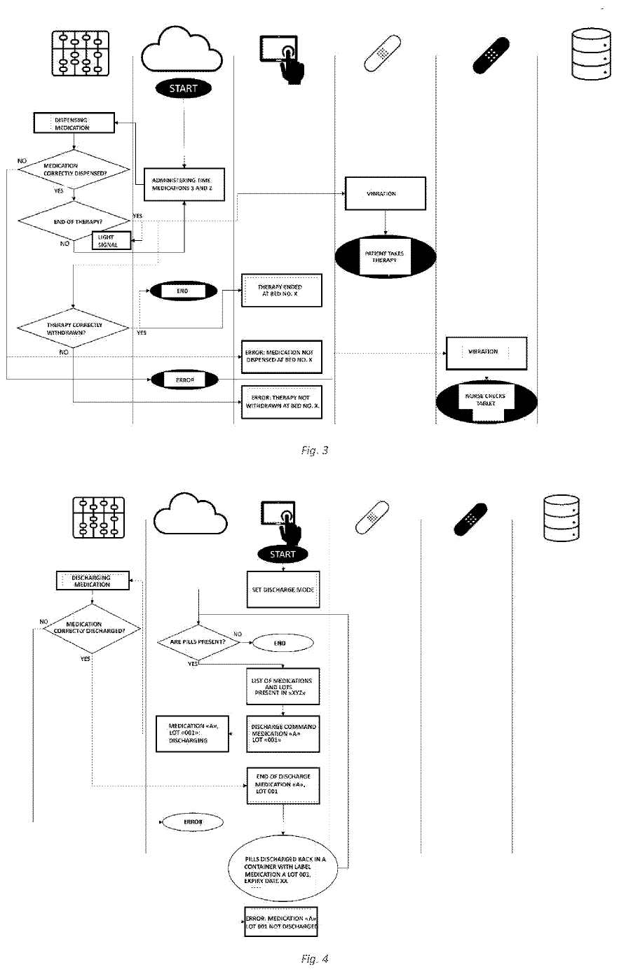

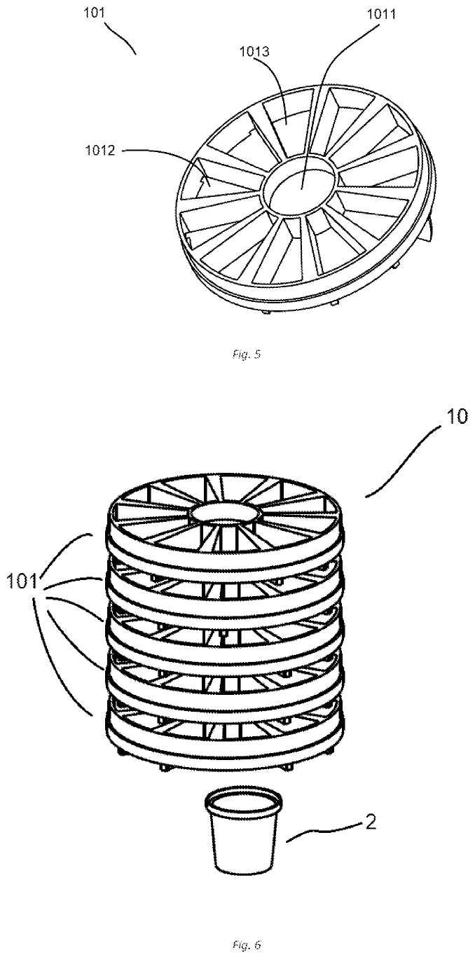

### DETAILED DESCRIPTION OF THE PREFERRED EMBODIMENTS

As it is shown in FIG. 5, the basic unit of the medication storage is made up of a rotating disc (101) comprising a central through-hole (1011) and an outer circular crown subdivided in a plurality of compartments (1012) of equal shape and dimensions, open on the top and provided, except one, with openable bottom. The compartment (1013) not provided with the openable bottom is completely bottomless and so is a through-hole inside the surface of the disc storage (101).

The bottom door (10121) of each compartment (1012) in installed on the bottom of the same to be able to rotate around a horizontal rotation axis (10122).

The door is normally in closed position, position in which it is kept by a spring system pushing it upwards. The door (10121) further comprises a lever (10123), which, when actuated by an outer element, causes the rotation of the door (10121) around the horizontal rotation axis (10122), thus opening the compartment (1012). When the outer action on the decoupling lever (10123) ends, a spring (not shown in figure) brings the door (10121) back in the closing position.

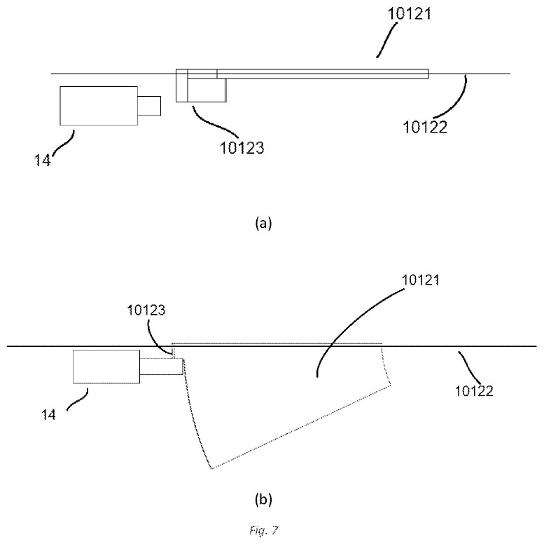

A plurality of discs (101) is stacked to provide a storage (10), as it can be observed in FIG. 6. The medication storage (10) is contained in a case (1) provided with a frontal opening (13) through which the medication storage (10) can be introduced inside said case (1) and an upper opening (12) through which the medications can be introduced inside the storage (10). The opening (13) to the medication storage is closed by a door (11), shown in opening position in FIG. 9.

The door (11), when closed, also allows the access to a medication withdrawing area (111), so that such area can be accessible also with the main door (11) closed. The device (1) further comprises automatic closing means of said medication withdrawing area (111) configured to prevent, when closed, the access to said area and to allow the access when opened.

The device (1) further comprises a plurality of actuators (14), one for each of the storage discs. Each actuator (14) is configured to actuate or not, as a function of its own position, the decoupling lever (10123) associated to the disc compartment that, in that moment, is near the same actuator.

FIG. 7-a shows an actuator (14) in distal position to the disc. The disc is not shown for clarity, and only the door (10121) is shown.

With the actuator in distal position, the lever in that position the disc can rotate without interference between the decoupling lever (10123) and the actuator (14).

FIG. 7-b shows an actuator in proximal position to the disc. With the actuator in this position, the decoupling lever (10123), after the disc rotation, intercepts the actuator (14). The rotation of the disc causes in this way the opening of the closing door (10121) of the compartment (1012). The actuator (14) associated to each disc is positioned so to intercept, when actuated, the decoupling lever (10123) of the compartment that in that moment is at the charge/discharge column.

The control means provided on the device are configured to actuate individually each actuator, selectively opening the compartment of each disc which is at the charge/discharge channel.

The opening sequence of the bottom compartment provides:
positioning of the disc with the compartment to be opened in the position adjacent to the one of the charge/discharge column, positioning of the respective actuator in position proximal to the disc,
rotation of the disc so that the compartment to be opened is at the charge/discharge column.

During the last disc rotation, the decoupling lever intercepts the actuator, and the disc movement makes the door (10121) rotate around the horizontal rotation axis (10122).

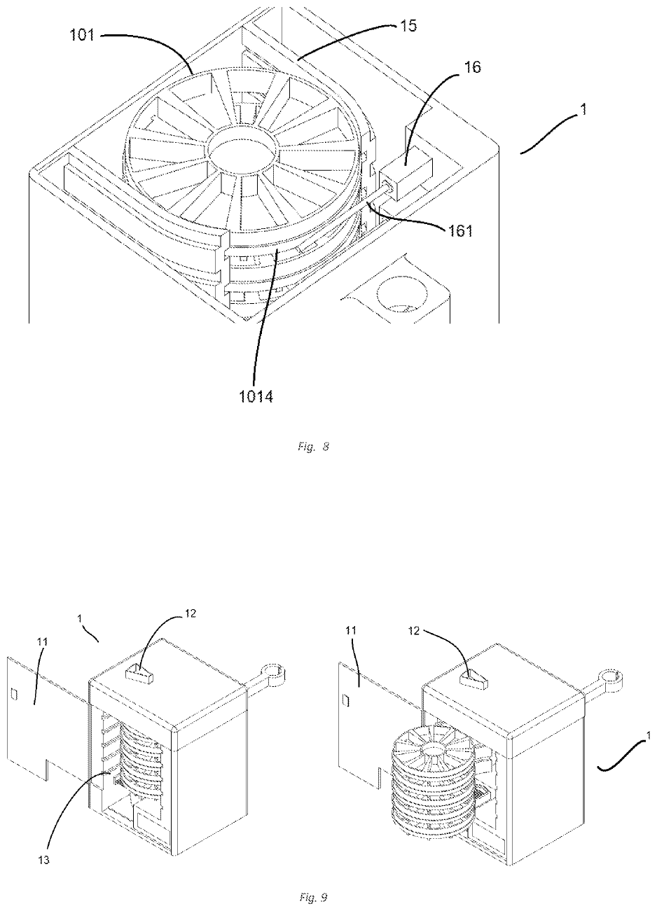

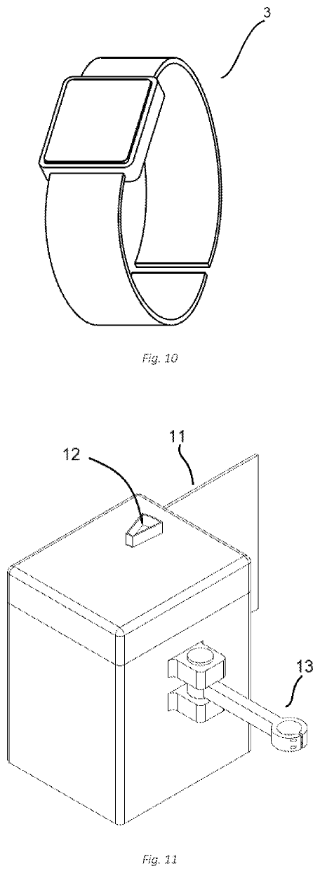

As it can be observed in FIG. 8, the shape of the housing (15) of each disc (101) is such that it allows an easy extraction by means of horizontal translation, in order to carry out its disinfection easily.

In the embodiment known shown in FIG. 5, each disc (101) is subdivided in 11 compartments (1012), anyway embodiments with a different number of compartments can be realized without departing from the aims of the invention.

Moreover, preferably, in order to optimize the usage of the space, the compartments (1012) will have the shape of circular crown sector.

Each compartment (1012) is configured to receive a single dose of a single medication, in loose pill, tablet or pre-cut blister form.

As it is shown in FIG. 6 and yet said, the device according to the invention comprises a plurality of discs (101), coaxially superimposed on each other and configured to be able to rotate to each other. Hence, the discs develop together the medication storage (10) in vertical direction.

FIG. 6 shows 5 different discs (101), anyway embodiments with a different number of discs can be used without departing from the aims of the invention.

The device further comprises an actuation system configured to allow the individual and independent rotation of each disc, and control means allowing to control such individual rotation means. Preferably the device comprises a plurality of electric motors (16), one associated to each disc (101) of the storage (10) and configured to make the respective disc rotate by means of a worm screw (161) acting on a specific toothed surface (1014) provided on the outer edge of the same disc.

In particular, the control means are configured to make each disc carry out multiple angular movements of the angle of each compartment (1012) by means of said actuation system.

Since in each disc (101) a single bottomless compartment (1013) is provided, it is to be observed that when the discs (101), coaxially superimposed on each other, are rotated so that all the bottomless compartments (1013) are in turn superimposed, a vertical passage channel is formed, in the following indicated also charge/discharge channel or charge/discharge line. When the discharge channel is aligned under the upper opening (12) of the case (1)—in a configuration identified in the following as basic configuration—and the closing means of said opening (12) are in position whereby said opening (12) is opened, it is possible to introduce medications inside the device. From the basic configuration, while maintaining all the actuators (14) in distal position and rotating a single disc so as to position a determined compartment in said disc under the opening (12), a configuration is obtained in which the medication introduced on the top inside the device falls in the compartment (1012) arranged under the opening (12). Thus, it is possible to charge a medication in any compartment (1012) of any disc (101).

It is in fact important to be underlined that each compartment can be identified individually through the number of disc it belongs to and the number of angular steps the disc has to carry out, with respect to the basic configuration, to bring that compartment at the charge/discharge channel. In other terms a couple of numbers identifies each storage compartment univocally.

The case (1) of the device, in the upper portion and at the opening of the charge/discharge channel, comprises an opening (12) covered by a movable cover provided with appropriate actuating means.

This allows to avoid the accidental introduction of pills, since control electronic means of the device will allow the opening of such movable cover only in the medication charging step.

After describing the disc embodiments, now their functioning can be explained in the charging step of the nth sector of the kth disc.

The storage (10) is brought in its basic configuration, in which all the discs are rotated up to bring their own bottomless compartment on the same line, thus realizing a charging line at the upper opening (12).

So, the kth disc is rotated so to position the nth compartment along the charging line. Since all the discs positioned thereon have their bottomless compartment along the charging line, the passage of the medication is guaranteed, which is introduced from the top through the specific opening provided on the case of the device.

It is to be specified that preferably, the device is configured to open and close the opening provided in the upper portion of the device before and after each single medication dose is charged, and to carry out movements of the storage disc, whether functional to the device charging or discharging, with all the openings of the case closed.

This measure reduces the possibility of device jamming or incorrect medications charging.

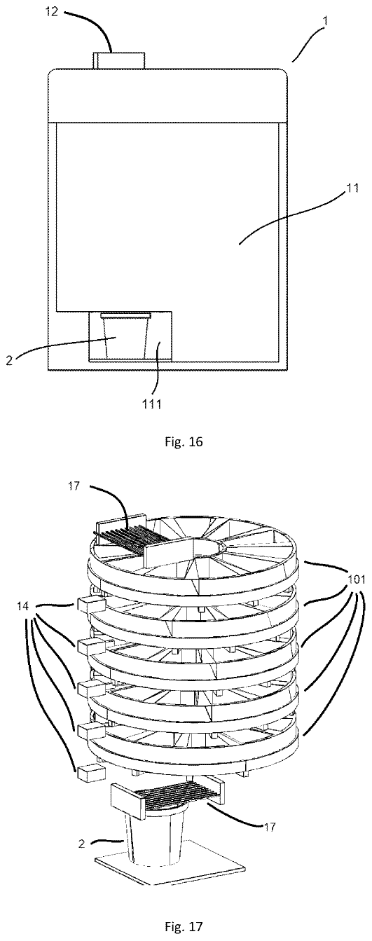

Similarly, the discharging step is carried out by bringing the compartment containing the pill to be dispensed in the position adjacent to the one at the discharge line, while all the discs under the same are rotated up to bring their own bottomless compartment on the same line. Then, the actuator (14) is actuated corresponding to the disc (101) on which the compartment (1012) to be opened is arranged, and in the following the compartment (1012) to be opened is brought, by rotating the disc, at the discharge line. This movement causes the opening of the bottom (10121) of the compartment (1012), and the medication contained thereinside falls along the discharge line, under which a container (2) is conveniently arranged for collection of discharged medications. As it can be observed in FIG. 16, the container is at the medication dispensing area (111), which is made accessible to the patient after that all the medications of the multi-medication dose are provided in the container (12).

The charge/discharge channels are appropriately sensorized in order to monitor the passage of pills.

In a preferred embodiment, photoelectric cells (17) are provided, positioned so to detect the passage of pills in the charging steps and photoelectric cells (17) positioned so to detect the passage of pills in the discharging steps. Preferably, the device further comprises a charging cell (18) configured to detect the variations of weight of the glass (2) as a function of its content. The control means of the device are configured so that the withdrawing of the container (2) inside the case is allowed only by moving the bracelet (3) worn by the patient close to the device (1), and after checking that the device brought close is the one associated to the patient who has to take the prepared dose. The closing means of the medication withdrawing area (111) are then unblocked, thus allowing the access to the dose withdrawing area.

The same bracelet (3), as yet said in the previous paragraph, is configured to signal the patient, by means of acoustic, light signaling or by vibrations, that in the device (1) a medication dose is provided, to be taken. Above all, it is the device that lights up/rings while the bracelet vibrates but it is also possible that the latter lights up or rings.

The medication withdrawing area is suitably sensorized in order to monitor the medication withdrawing by the patient.

In a first embodiment, the device comprises a charging cell (18) detecting the weight of the container (2) in which the medication is dispensed to the patient. Conveniently, the device is also configured to calibrate said charging cell before the dispensing step starts and to weigh again the container put back by the patient after dispensing, checking the real emptying of the same by weighing. In another embodiment, the device comprises a housing for the container (2) which allows the positioning of the glass only if aligned with said housing.

The housing can be rotated by 180° by means of the control means of the device, so that in the repositioning step of the container (2), the same can be necessarily positioned with the opening downwards, to guarantee the emptying thereof.

Yet, in another embodiment, the device comprises a camera configured to frame the bottom of the container (2), and the device comprises control electronic means and respective computer programs configured to analyze the image of the container individuating the presence of medications thereinside.

The access area will be closed again once the patient has correctly repositioned the empty glass. Moreover, preferably, but not limitingly, the device (1) comprises a button accessible from outside, and the device is configured to deactivate said acoustic or light signaling means only after that the container (2) has been put back in its place and the patient has actuated said button, thus confirming the successful administering of the dispensed medications.

As an alternative, the device is configured to send a message of medication administering check to the device wearable by patient after the withdrawing of the container (2), and to deactivate said light or acoustic signaling means only after the patient, by means of said wearable device, has sent an answer confirming the successful administering of the dispensed medications.

### Deblistering Device

In the following, a preferred, but not limiting, embodiment of the device is described, further comprising a deblistering unit, configured to process whole blisters of medications without limitations of dimensions and type of medications. The deblistering unit (200) comprises:
a pick and place system (201),
an extraction station of pills (202).

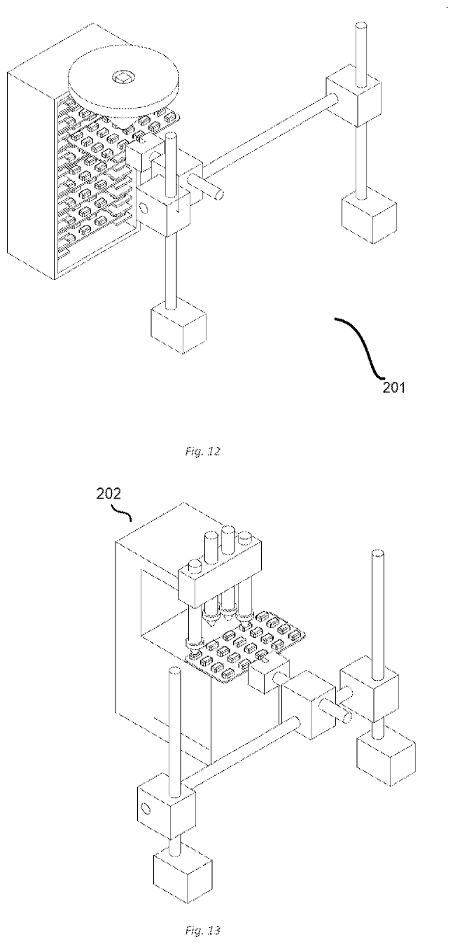

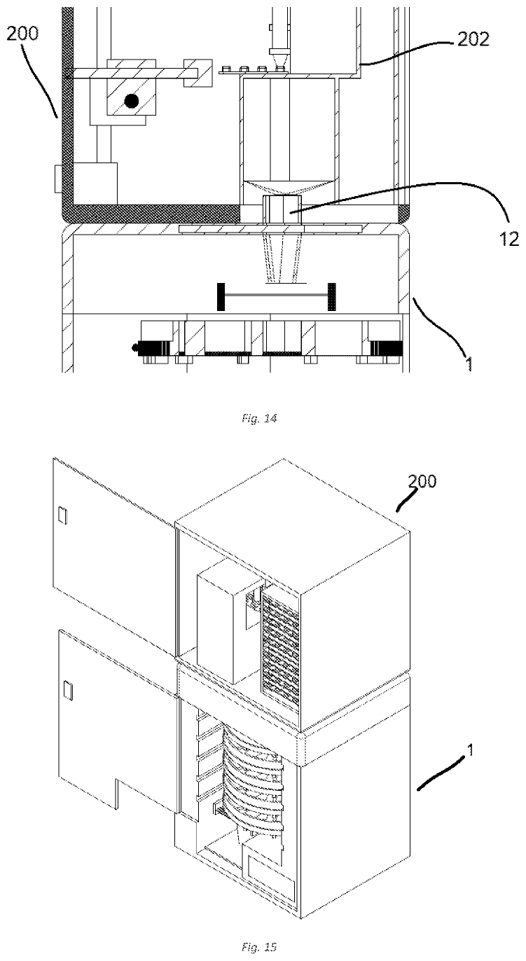

The pick and place system, shown in FIG. 12, is configured to extract the blisters stored in a storage and to position them in the working area of a display system, which identifies the position of the single pills in the blister. The blister is then positioned in the extraction station of pills shown in FIG. 13. The system acts mechanically on the blister by extracting the single pill with a system of actuators having dimensions and shape adapted to the medication ones. The extracted pills are conveyed in the introduction channel of the device, as it is shown in FIG. 14. FIG. 15 shows the deblistering device assembled on the device.

According to what said, the device is configured so to produce a multi-medication dose (i.e. administering of a plurality of medications together) autonomously and in automatized manner.

Inside the device, the different medications are in fact stored singularly in separated compartments and discharged each time in a dispensing compartment at the moment of the planned administering or of the administering on demand.

The device comprises computing and control electronic means and a user interface, which allow: in charging step, to associate to each compartment the respective medication stored therein, in using step, to provide in automatic and automatized manner the dose of medications to be administered to the patient.

At the planned time, the device prepares the dose (single-medication or multi-medication) by making all the medications, which are to be taken, fall in the dispensing compartment.

Once the dose has been discharged inside the compartment, the signaling means indicate to the user that the dose is ready to be taken.

In a first embodiment, the signaling means comprise warning lights, in other embodiments the signaling means comprise acoustic signaling means, as for example the reproduction of a sound or a prerecorded voice.

In a preferred embodiment, the device is provided with remote wireless communication means and near field communication means (NFC) , and further comprises at least a device wearable by the patient, provided with respective remote wireless communication means and near field communication means, in addition to signaling means, as for example means for the vibration of the same. Preferably, the device comprises a plurality of wearable devices, each assigned to a specific patient.

In this embodiment, the device is configured to actuate the signaling means of the wearable device associated to the patient who has to take the dispensed medication dose, to inform the latter that it is needed to take the dose. The device further comprises closing means of the compartment where the dose to be taken is discharged, and said control means are configured to allow the opening of said closing means upon identification, by means of said near field communication means, of the correct wearable device. This allows to make sure that the patient is present, who has to take the dose at the moment in which it is provided, thus substantially reducing the risk of incorrect administering.

In other terms, upon actuation of the signaling means, in order to access to the dose the patient has to move his own wearable device (for example a bracelet) close to the NFC reader provided on the device, which will allow access to the dose after identifying the wearable device.

Preferably, the device is configured so that the reading of the wearable device interrupts the output of acoustic warnings and the bracelet vibration, while the warning lights on the same remain on until the dose is withdrawn from the compartment in which it has been dispensed.

Moreover, preferably, the device is configured to turn off the warning lights only after that the removable container, where the dose has been dispensed, is put back in the dispensing compartment.

When the procedure is ended, the light signal turns off and the access to the dispensing compartment is closed.

The device is also configured to allow the dispensing of “on demand” or “at need” therapy, for those medications that do not have to be taken at predetermined times, but after the appearance of specific symptoms.

To this purpose, the device is provided with at least a button, or any other equivalent means to allow the patient to make a command.

In this way, for dispensing the “at need” therapy, the patient moves autonomously the bracelet close to the reader, and presses the button for dispensing the pill at need.

Also in this case, the light signal turns on after the medication dispensing, and the signal remains on until the patient has withdrawn the dose, and, if planned, has put the glass back in the administering compartment, then closed again the access to the administering compartment.

As yet said in the introduction, one of the characteristics of the device according to the present invention is the possibility to use in addition to “loose” medications also “blistered medications, i.e. still sealed in their own blister.

The medication dispensing system comprises at least a device of the just described type (and preferably a plurality of devices of the just described type), and a main electronic device allowing its management, on which computer programs are loaded that allow the management of all the associated devices.

Possibly, the management software can be loaded on remote control means, to which the main electronic device can access by authentication by intranet or web.

The main electronic device comprises a barcode reader (possibly implemented through a camera and software means allowing the identification of the framed barcode), so that in the medication charging step the medication in the charging step inside the system can be univocally identified.

Moreover, preferably, the main electronic device comprises a QR code reader, so to acquire in automatized manner in addition to the type of the medication, also additional information, such as the lot number and the expiry date, if such information is available on the medication package. It is now described the charging procedure of the medications inside the device.

This procedure can be carried out, by means of the main electronic device, by an authorized user (typically a nurse) having the suitable access credentials. Once logged, by means of the barcode reader, the authorized user associates the device according to the invention (which is provided with a univocal identification code) to at least one wearable device (provided as well with an identification code) univocally associated to a patient.

In this way, a biunivocal correspondence between patients and wearable devices is defined, and a correspondence between one to many between dispensing device and wearable devices associated to the patients.

At this point, the authorized user sets, by means of the interface of the main electronic device, the prescribed therapy for each patient.

It is to be observed that such therapy can be alternatively imported automatically by management software of the hospital ward, if it has been input by a physician, or manually input by the authorized user.

These computer implementation aspects will be able to be modified without departing from the aims of the invention.

Since, manually or automatically, the indication relating to the dosage quantity and frequency has been provided-for each medication, it is now known the type and whole quantity of each medication needed to administer the therapies to the patients associated to the dispensing device.

At this point, the authorized user can recognize the medication by reading the barcode or QR.

Upon identification of the medication, the software displays on the user interface the name of the medication, positions the first compartment to be charged at the charging channel and in the following opens the upper opening for the charging of the dispensing device, thus allowing the nurse to charge a dose of medication, and then closes again the upper opening.

The nurse charges, in the individual spaces assigned thereto, all the medications (pills, tablets or else) planned by the therapy of the patients associated to the dispensing device, and inputs the information relating to the production lot and expiry date of the medication.

When this operation has been accomplished, the software closes the access to the first charging compartment and gets ready for charging the second medication and the next ones, which occur at the same way as the just described ones.

It is to be observed that charging the single administering of different medications in separate spaces, and each type of medication inside a separated compartment, allows the recognition (and so the re-usage) of the single medication (and respective production lot and expiry date) in case of emptying the device for a therapy interrupted before planned or modified during the stay in hospital, which are very often occurring circumstances. So, the possibility to reuse the medications charged in the device, but not used, represents a great cost saving for the hospital.

The charging procedure of the device, in case of a new therapy or modification of the same, can be carried out also when the dispensing device is yet in the room of the patient, by using a portable main electronic device (for example a hand-held computer or a tablet).

The dispensing device is configured to communicate to the main electronic device, by sending warnings in at least one of the following situations: unsuccessful withdrawing by the patient of the dispensed dose, unsuccessful completion of the dose administering procedure, jamming, discharged battery, lack of signal, lack of pills, exhausted sim, errors of any nature and lack of pills to be dispensed.

In a preferred embodiment, the system further comprises a wearable device (for example a bracelet) given to the health care professional supervising the hospital ward (the nurse on shift), provided with remote communication means and configured to receive said warnings.

Furthermore, preferably, the device comprises a visual signaling means (warning light) which is activated when one of the just described situations occurs. Preferably said warning light is of color other than the warning light turning on to warn about the availability of the dose to be dispensed. The dispensing device is further configured to communicate to the main electronic device, by sending warnings at each single successful medication dispensing.

The computer programs loaded on said main device are configured to provide to the staff reports about the quantity of administering carried out for each patient (administering occurred on time, delayed, skipped . . . ), in addition to reports allowing the traceability of the users who have worked on the system and the single charging operations carried out.

The dispensing device according to the invention has conveniently reduced dimensions, with a base of approximately maximum 20 cm width and 15 depth, and a height approximately within 30 cm, so that it is easy to position the same on a bedside table.

The device further comprises removable anchorage means, which allow the anchorage thereof on a horizontal plane (for example suction cups) or to the bed of patient or other support.

The device further comprises supplying means by battery, or by electric network, and preferably further comprises a back-up battery intervening in case the main battery is discharged or in case of lack of network tension.

1. A device for medication automatic storage and dispensing, comprising:
a medication storage (10) comprising a plurality of discs (101), coaxially superimposed on each other and configured to be able to rotate to each other, each disc (101) comprising:
a plurality of compartments (1012) open on the top;
a case (1) provided with a frontal opening (13) protected by a door (11) through which said medication storage (10) can be introduced inside said case (1),
an actuation system configured to allow the individual and independent rotation of each disc;
and wherein
said compartments (1012) are provided with openable bottom, and each of said discs comprises a bottomless compartment (1013), so that when said bottomless compartments (1013) are superimposed on each other, a medication charge/discharge channel is obtained, passing from the top to the bottom of said storage (10),
and wherein,
said case (1) comprises an upper opening (12), through which the medications can be introduced inside said storage (10);
and
wherein said device further comprises:
a removable collection container (2) of medications to be dispensed to the patient positioned inside said case, under said medication storage (10) and at said upper opening (12), said door (11) being configured so as to allow, even when it is closed, to extract said container (2) through a medication access area (111); automatic protection device of said medication access area (111) and said upper opening (12);
control unit configured to control the opening of said automatic protection device of said medication access area (111) and of said upper opening (12), said actuation system for the discs rotation and the opening of said compartments (1012) provided with openable bottom.

2. The device according to claim 1, wherein a bottom door (10121) of each of said compartments (1012) is mounted so that it can rotate around its own horizontal rotation axis (10122), it is kept in closed position by a spring system and further comprises a lever (10123) that, when actuated by an outer element, causes the rotation of the door (10121) around its own horizontal rotation axis (10122), thus opening the compartment (1012).

3. The device according to claim 1, further comprising a plurality of actuators (14), one for each of said storage discs, each actuator (14) being movable between a first and a second position and being configured to actuate, as a function of its own position and after the rotation of the disc it is associated to, said decoupling lever (10123) associated to the compartment of the disc rotating at the same actuator.

4. The device according to claim 1, wherein said control unit is configured to carry out a sequence for opening the bottom of one of said compartments, comprising the steps of:
positioning of the disc with the compartment to be opened in the position adjacent to the one of said charge/discharge channel,
positioning of the respective actuator in position proximal to the disc,
rotation of the disc so that the compartment to be opened is at the charge/discharge column and during the rotation said decoupling lever intercepts the actuator, and the movement of the disc makes the door (10121) rotate around the horizontal rotation axis (10122).

5. The device according to claim 4, wherein said compartments (1012) have the shape of circular crown sector.

6. The device according to claim 1, wherein each compartment (1012) is configured to receive a single dose of a single medication, in loose pill, tablet or pre-cut blister form.

7. The device according to claim 1, wherein said actuation system comprises a plurality of electric motors (16), one associated to each disc (101) of the storage (10) and configured to make the respective disc rotate by means of a worm screw (161) acting on a specific toothed surface (1014) provided on the outer edge of the same disc.

8. The device according to claim 1, wherein said charge and discharge channel comprises sensors apt to detect the passage of the pills charged in the device or discharged by the same.

9. The device according to claim 8, wherein said sensors comprise photoelectric cells (17), positioned so to detect the passage of pills in the charging steps and photoelectric cells (17) positioned so to detect the passage of pills in the discharging steps.

10. The device according to claim 1, further comprising a charging cell (18) configured to detect the variations of weight of said removable container.

11. The device according to claim 1, further comprising a remote wireless communication unit and a near field communication unit.

12. An integrated system for medication dispensing, comprising:
at least a device according to claim 1,
at least a wearable device (3), univocally associated to a respective patient and provided with wireless communication devices and acoustic, a light signaling device or vibrating device;
at least a main control and management electronic device for the management of the system;
computer programs, loaded on a respective processor unit and accessible by remote communication, and configured to manage the devices provided in the system.

13. The system according to claim 12, further comprising at least a wearable device associated to a health care professional.

14. The system according to claim 12, wherein said control unit of each device (1) is configured so that said automatic protection device of said medication access area (111) are unblocked, thus allowing the withdrawal of said container (2) only by moving the wearable device (3), associated to the patient who has to take the prepared dose, to the device (1).

15. The system according to one of claim 12, wherein said device comprises sensors configured to check the actual emptying of said container (2) by the patient.

16. The system according to claim 15, wherein said at least one device comprises a charging cell (18) configured to detect the weight of the container (2) in which the medication is dispensed to the patient, in that the device is also configured to calibrate said charging cell before the dispensing step starts and to weigh again the container put back by the patient after dispensing, checking the real emptying of the same by weighing.

17. The system according to claim 15, wherein said device comprises a housing for the container (2) which allows the positioning of said glass only if aligned with said housing, in that said housing can be rotated by 180° by means of the control unit of the device, wherein said control means unit is configured so that, in the repositioning step of the container (2), the same is to be necessarily positioned with the opening downwards, to guarantee the emptying thereof.

18. The system according to claim 15, further comprising a camera configured to frame the bottom of the container (2), and the device comprises a control circuitry and respective computer programs configured to analyze the image of the container individuating the presence of medications thereinside.

19. The system according to one of claims 12, further comprising a deblistering unit, which can be positioned on said device and comprising:
a pick and place system, configured to extract the blisters stored in a storage and to position them in the working area of a display system, which identifies the position of the single pills in the blister;
an extraction station of pills, comprising a plurality of actuators configured to extract a single dose of medication and to make it fall, through said opening (12), in said medication storage (10).

20. The system according to claim 12, to wherein inside said at least one device the different medications are in fact singularly stored in separated compartments and further comprising a processor unit and control circuitry and a user interface, allowing to associate to each compartment the respective medication stored thereinside, in the charging step.

21. The system according to claim 20, characterized in wherein said processor unit and control circuitry are configured so that, at the planned time for the administering of one or more medications, the device makes all the medications, which are to be taken, fall in said removable container (2), and so that, after the all administering has been discharged inside said container (2), the light signaling device provided on said wearable device (3) indicates to the patient that the dose is ready to be taken.

22. The system according to claim 12, wherein said device is further configured to allow the dispensing of “on demand” therapy, for those medications that do not have to be taken at predetermined times but after the appearance of specific symptoms, and comprising a at least a button, or any other equivalent ways to allow the patient to make a command, and is configured to dispense a medication upon actuation of said command by the user.

23. The system according to claim 12, wherein said device (1) comprises a button accessible from outside, and is configured to deactivate said acoustic or light signaling device only after that the container (2) has been put back in its place and the patient has actuated said button, thus confirming the successful administering of the dispensed medications.

24. The system according to one of claim 12, wherein said device (1) is configured to send a message of medication administering check to the device wearable by the patient (3) after the withdrawing of the container (2), and is further configured to deactivate said light signaling device or acoustic signaling device only after the patient, by means of said wearable device (3), has sent an answer confirming the successful administering of the dispensed medications.
